SEQUENCE LISTING (4 sequences)

SEQ ID NO: 1 - protein, 5 aa, Homo sapiens
  MOD_RES (5)..(5): AMIDATION
FFGLM

SEQ ID NO: 2 - protein, 4 aa, Homo sapiens
  MOD_RES (4)..(4): AMIDATION
FGLM

SEQ ID NO: 3 - protein, 11 aa, Homo sapiens
  MOD_RES (11)..(11): AMIDATION
RPKPQQFFGLM

SEQ ID NO: 4 - protein, 5 aa, Homo sapiens
  MISC_FEATURE (2)..(2): “X” may be either F or V
FXGLM

(19) United States
(12) Patent Application Publication
(10) Pub. No.: US 2004/0171093 A1
(43) Pub. Date: Sep. 2, 2004

(54) Methods for detecting deficient cellular membrane tightly bound magnesium for disease diagnoses

(76) Inventor: Ibert C. Wells, Omaha, NE (US)

Correspondence Address: STINSON MORRISON HECKER LLP, ATTN: PATENT GROUP, 1201 WALNUT STREET, SUITE 2800, KANSAS CITY, MO, 64106-2150, US

(21) Appl. No.: 10/805,881

(22) Filed: Mar. 22, 2004

Related U.S. Application Data
(63) Continuation-in-part of application No. 10/053,669, filed on Jan. 24, 2002.
Application No. 10/053,669 is a continuation of application No. 09/265,690, filed on Mar. 10, 1999, now Pat. No. 6,372,440.
(63) Continuation-in-part of application No. 10/695,536, filed on Oct. 28, 2003.
Application No. 10/695,536 is a division of application No. 10/230,133, filed on Aug. 29, 2002, now Pat. No. 6,664,420.
Application No. 10/230,133 is a division of application No. 09/635,266, filed on Aug. 9, 2000, now Pat. No. 6,455,734.

(51) Int. Cl. G01N 33/53; G01N 33/537; G01N 33/543; C12Q 1/68

(52) U.S. Cl. 435/7.92

(57) ABSTRACT

This invention relates to methods for detecting the deficiency of magnesium tightly bound to plasma membranes of somatic cells, referred to as the magnesium binding defect. The invention also relates to methods for assessing certain abnormal physiological states, such as, salt-sensitive essential hypertension, type 2 overt or prediabetes mellitus, and preeclampsia/eclampsia syndrome that are associated with the magnesium binding defect. The invention further relates to methods for generating magnesium deficient cell membranes and for identifying substances which promote binding of magnesium ions to the plasma membranes of somatic cells. Additionally, the invention relates to a binding pair members having affinity for the peptides and promoters of the invention.

This application is a continuation-in-part of U.S. patent application having Ser. No. 10/053,669 filed Jan. 24, 2002, which is a continuation of U.S. patent application having Ser. No. 09/265,690, filed Mar. 10, 1999, now U.S. Pat. No. 6,372,440, all of which are incorporated herein. This application is also continuation-in-part of U.S. patent application having Ser. No. 10/230,133, filed Aug. 29, 2002, which is a divisional of U.S. patent application, having Ser. No. 09/635,266, filed Aug. 9, 2000, now U.S. Pat. No. 6,455,734, the entirety of the disclosures of which are hereby specifically incorporated herein by reference.

### BACKGROUND OF THE INVENTION

The present invention relates to methods for detecting the magnesium binding defect (MgBD) in the plasma membrane of somatic cells, which defect is critically associated with physiological disorders, such as salt-sensitive essential hypertension, type 2 diabetes mellitus (both pre- and overt stage) and preeclampsia/eclampsia syndrome. More specifically, the present invention relates to the detection of the magnesium binding defect for assessing a predisposition to one or more of such disorders and the management thereof. The present invention further relates to a method for identifying substances which promote binding of magnesium ions to the plasma membranes of somatic cells and thereby correct the MgBD. The present invention also relates to a method for generating magnesium deficient cells. The present invention still further relates to a binding pair member, such as an antibody, having affinity for one or more of the peptides of the invention.

The publications and other materials used herein to illuminate the background of the invention, and in particular cases, to provide additional details respecting the practice, are incorporated by reference and for convenience are referenced in the following text by author and date and are listed alphabetically by author in the appended List of References.

Hypertension is a leading cause of human cardiovascular morbidity and mortality, with a prevalence rate of 25-30% of the adult Caucasian population of the United States (JNC Report 1985). The primary determinant of essential hypertension, which represents 95% of the hypertensive population, have not been elucidated in spite of numerous investigations undertaken to clarify the various mechanisms involved in the regulation of blood pressure. Although there are exceptions, most untreated adults with hypertension will continue to experience further increases in their arterial pressure over time. Reports based on actuarial data and clinical experience, estimate that untreated hypertension shortens life by 10 to 20 years. This lower life expectancy is believed to be due to an acceleration of the atherosclerotic process, with the rate of acceleration related in part to the severity of the hypertension. Even individuals with relatively mild disease, e.g., those individuals without evidence of end-organ damage, if left untreated for 7 to 10 years have a high risk of developing significant complications, and more than 50 percent of them will ultimately experience end-organ damage related to hypertension. End organ damage can include cardiomegaly, congestive heart failure, retinopathy, a cerebrovascular accident, and/or renal insufficiency. Thus, even in its mild forms, hypertension can be a lethal disease, if left untreated.

Although awareness of the problems associated with elevated arterial pressure has increased, the cause of the disease, and thus potentially its prevention and cure, is still largely unknown. These individuals have only generalized or functional abnormalities associated with their hypertension and are often diagnosed as having primary, idiopathic or essential hypertension. Several abnormalities have been identified in patients with essential hypertension (Meyer and Marche, 1988), often with claims, later contested or unsubstantiated, of the abnormalities being primarily responsible for the hypertension. This situation has been attributed generally to the likely possibility that essential hypertension has more than one cause, each of which may be a set of genetically determined, contributory abnormalities, which in turn interact with environmental factors.

The most widely recognized of these possible causes of essential hypertension is sodium ion (Na+)sensitivity, also commonly referred to as salt (NaCl)-sensitivity. In some patients with essential hypertension the hypertension is exacerbated by a high dietary salt intake and diminished by dietary salt restriction. It has been assumed that this abnormality reflects a cellular membrane defect, and that this defect occurs in many, perhaps all, cells of the body, particularly the vascular smooth muscle cells. Based on studies using erythrocytes, this defect has been estimated to be present in 35 to 50 percent of the essential hypertension population.

Type 2 diabetes mellitus is the most common form of diabetes mellitus, comprising 85-90% of the diabetic population and taking heterogeneous forms. The symptomatic stage (overt) of type 2 diabetes mellitus characteristically appears after age 40, has a high rate of genetic penetrance unrelated to genes of the human major histocompatibility complex (HLA), and is associated with obesity. A strong hereditary component is evident. For example, concordance rates in identical twins is nearly 100 percent.

Among Caucasian Americans the estimated incidence of type 2 diabetes mellitus in 1976 was between 1 and 2 percent. However, the prevalence has risen as the population has aged and become more obese, and currently more than 10 percent of the older population suffers from the disease. According to the 1990-1992 National Health Interview Survey, about 625,000 cases of type 2 diabetes are diagnosed in the United States each year. This is more than 6 times the 1935-36 rate.

Many consider insulin resistance to be the primary cause of type 2 diabetes mellitus. This insulin resistance and the consequent hyperinsulinemia are evident years before insulin secretion diminishes and overt diabetes mellitus is present. These two pathophysiological processes are embodied in type 2 prediabetes mellitus. About 20 percent of the Caucasian population of the United States has impaired glucose tolerance, i.e. hyperglycemia, the virtually universally accepted sign of the presence of overt diabetes mellitus.

Patients affected with overt type 2 diabetes mellitus retain some endogenous insulin-secreting capacity, but insulin levels in plasma are low relative to the magnitude of insulin resistance and ambient plasma glucose levels. Such patients do not depend on insulin for immediate survival and rarely develop diabetic ketosis.

The clinical presentation of type 2 diabetes mellitus is insidious. The classical symptoms of diabetes may be mild and tolerated for a long time before the patient seeks medical attention. Moreover, if hyperglycemia is asymptomatic, the disease becomes clinically evident only after complications develop. Such complications include atherosclerosis, the risk for which is greatest in poorly controlled patients. Other sequela of diabetes mellitus are myocardial infarction, stroke, peripheral vascular disease and lower extremity gangrene, neuropathy, nephropathy, diabetic foot syndrome, cardiomyopathy and dermopathy.

Little is known about the specific genetic abnormalities associated with most forms of type 2 diabetes mellitus. However, as reported herein, the magnesium binding defect was observed in the erythrocyte membranes of all mildly affected type 2 diabetics. For example, of twenty-four unmedicated, normotensive, type 2 diabetics examined, all possessed the defect.

It has been reported previously that insulin resistance is caused by the decreased concentration of tightly-bound magnesium in the plasma membrane of somatic cells, referred to as the magnesium binding defect (Mattingly et al., 1991). These observations strongly support the concept that the magnesium binding defect, which has a genetic origin, is the cause of insulin resistance. The mechanism involved and reversal of the defect by an unidentified component of normal plasma is discussed in Wells and Agrawal (1992).

Preeclampsia/eclampsia syndrome is a member of a group of hypertensive disorders related to the common medical complications of pregnancy. In 1972 the American College of Obstetricians and Gynecologists recommended classification of hypertension during pregnancy into chronic hypertension, preeclampsia, preeclampsia super imposed on chronic hypertension, and transient hypertension. Subsequently, two international committees provided slightly different definitions for preeclampsia. The International Society for the Study of Hypertension in Pregnancy defined preeclampsia as hypertension and proteinuria developing during pregnancy, labor, or puerperium in a previously normotensive nonproteinuric woman (Davey and MacGillivray, 1988). Preeclampsia has been defined by the National High Blood Pressure Education Program Working Group as increased blood pressure accompanied by proteinuria, edema, or both (Gifford et al., 1990). These diverse definitions arose because the etiology of preeclampsia has heretofore remained unknown and, prior to the results reported herein, no definitive diagnostic sign or symptom has been identified.

Preeclampsia, a pregnancy-induced syndrome in humans which affects virtually all maternal organ systems, has been recognized from antiquity. It remains to the present day a major cause of maternal and perinatal mortality and morbidity. The etiology of this syndrome is unknown and its pathogenesis remains unclear; no specific diagnostic and/or prognostic tests have been reported. The syndrome is progressive and incurable except by the termination of the pregnancy, after which the pathophysiology regresses It is estimated that 7-10 percent of all pregnancies, worldwide, are affected and that preeclampsia accounts for some 200,000 maternal deaths per year. The syndrome has significant implications for the ongoing health of both mother and baby and is the most prevalent cause of maternal death in the United States of America, Scandinavia, Iceland, Finland, and the United Kingdom of Great Britain and Northern Ireland. Preeclampsia is considered to have a genetic component although seemingly contradictory observations are recorded in the relevant literature. Undoubtedly, a major reason this unique syndrome has remained an enigma is that it does not occur, nor has it been wholly induced, in experimental animals.

Because the etiology has remained unclear, there are no known diagnostic tests specifically for preeclampsia. Instead, the disorder is recognized by the occurrence of pregnancy induced changes that regress after delivery, of which hypertension and proteinuria are the easiest to recognize and are the signs by which the syndrome has heretofore been defined.

Thus, it was an object of the present invention to identify the substances in normal blood plasma which promote the binding of magnesium ion to plasma membranes of somatic cells and thereby ameliorate or correct the magnesium binding defect. Another object of the present invention was to utilize the discoveries of these substances to evaluate an association of the magnesium binding defect with physiological disorders, such as salt-sensitive essential hypertension, type 2 diabetes mellitus and preeclampsia/eclampsia syndrome. Yet another object of the present invention was to utilize such association to identify persons who may be predisposed to such disorders leading to better management of the diseases. It was a further object of the present invention was to develop a method to identify other compounds which promote the binding of magnesium ion to plasma membranes of somatic cells.

### SUMMARY OF THE INVENTION

The present invention relates to methods for assessing a predisposition to physiological disorders, such as: sodium-sensitive (salt-sensitive) essential hypertension; type 2 overt and prediabetes mellitus associated with the MgBD; and preeclampsia/eclampsia syndrome. The subnormal binding of magnesium to plasma membranes of the somatic cells is critically associated with an individual's susceptibility to develop such disorders. More specifically, the present invention has identified amidated peptides in blood plasma which are associated with the magnesium binding defect, and therefore, useful in the practice of the present invention. These armidated peptides characterize the amidated C-terminal amino acid sequences of all tachykinnins, of mammalian origin, i.e., Phe-X(Phe,Val)-Gly-Leu-Met-NH₂. It has been discovered as reported herein, that the determination of the level of these amidated peptides in blood plasma of an individual can identify individuals having such physiological disorders, as well as those with a predisposition to develop such physiological disorders. As a result, the treatment of the disorders in these subjects can then be more specifically managed, for example, in the case of salt-sensitive essential hypertension, by dietary sodium restrictions.

The present invention further provides a method to identify substances which promote the binding of magnesium ion to plasma membranes of somatic cells and can therefore be used to ameliorate or correct the magnesium binding defect. The present invention still further provides a method for generating somatic cells deficient in magnesium tightly bound to the plasma membrane. The present invention also provides a binding pair member, such as an antibody, having affinity for one or more of the peptides of the invention.

### SUMMARY OF THE SEQUENCES

SEQ ID NO:1 is the amino acid sequence of the pentapeptide of the present invention which is amidated.

SEQ ID NO:2 is the amino acid sequence of the tetrapeptide of the present invention which is amidated.

SEQ ID NO:3 is the amino acid sequence of Substance P which is amidated.

SEQ ID NO:4 is the generalized amino acid sequence of the amidated C-terminal end of all tachykinins of mammalian origin.

### DETAILED DESCRIPTION OF THE INVENTION

The present invention is directed to the determination of the substances in mammalian blood plasma which ameliorate or correct the magnesium binding defect in the plasma membranes of somatic cells. The discovery of these substances has made possible the development of a method for detecting the presence of the magnesium binding defect (MgBD). It has been found that the defect is critically associated with physiological disorders, such as salt-sensitive, essential hypertension; type 2 diabetes mellitus; and preeclampsia/eclampsia syndrome. Thus, the discovery of a method to detect MgBD makes possible a method for assessing a predisposition in an individual to these disorders. The association of the MgBD with preeclampsia and the predisposition to preeclampsia has been discovered as reported herein, thus permitting individuals at risk to be identified, treated and their treatment monitored. The further discovery of a method for generating magnesium deficit cells reported herein has made it possible to identify promoters of magnesium binding and monoclonal antibodies thereto.

Definitions. The present invention employs the following definitions:

“Magnesium Binding Defect” and “MgBD” refer to significantly less than normal levels of Mg²⁺ tightly bound to plasma membranes of somatic cells, which levels are not the result of a nutritional deficiency of magnesium.

The term “peptide mimetic” or “mimetic” is intended to refer to a substance which has the essential biological activity of SEQ ID NO:2, SEQ ID NO:1, or SEQ ID NO:4. A peptide mimetic may be, but is not limited to, a peptide containing molecule that mimics elements of protein secondary structure (Johnson et al., 1993). The underlying rationale behind the use of peptide mimetics is that the peptide backbone exists chiefly to orient amino acid side chains in such a way as to facilitate molecular interactions, such as those of antibody and antigen. A peptide mimetic is designed to permit molecular interactions similar to the natural molecule. A mimetic may not be a peptide at all, but it retains the essential biological activity of the natural peptide.

The term “Plasma” refers to proteinaceous fluid in which blood or lymph cells (the formed elements) are suspended.

“Preeclampsia” and “Preeclampsia/Eclampsia Syndrome” refer to hypertension plus hyperuricemia or proteinuria and have historically been categorized as mild or severe on the basis of degree of elevation in blood pressure and/or the degree of proteinuria (Sibai, 1996). Preeclampsia and preeclampsia/eclampsia syndrome may include one or more of a cluster of associated patho/physiological states including cerebral hemorrhage, eclampsia, cortical blindness, hepatic rupture, disseminate intravascular coagulation, pulmonary edema, laryngeal edema, acute cortical necrosis, abruptio placenta intrauterine fetal asphyxia and death.

The term “Serum” refers to blood plasma without clotting factors.

“Salt-sensitive” and “sodium-sensitive” essential hypertension refer to hypertension that is exacerbated by the ingestion of more than required levels of salt (NaCl), and that may be diminished by reducing dietary intake of NaCl and/or other sources of sodium ion.

“Type 2 diabetes mellitus” refers to glucose intolerance, caused by either or both of insulin resistance and decreased insulin secretion.

“Tightly bound magnesium” refers to magnesium bound to the plasma membrane of somatic cells, that is not found in the supernatant from lysed and washed cells, such lysing and washing procedure as further described herein. Tightly bound magnesium is involved in movement of Mg⁺ into the cell.

In one aspect of the present invention, methods for assessing a predisposition to, and for monitoring the progess of treatment, of abnormal physiological states associated with the magnesium binding defect is provided. The methods include measuring the level of one or more of the disclosed peptides in blood plasma or other body fluids and comparing the level to a standard, wherein a significantly lower level of peptide is indicative of the presence of the magnesium binding defect. In one embodiment of this aspect, the abnormal physiological state is the presentation of preeclampsia during pregnancy. In another embodiment, the abnormal physiological state is salt-sensitive essential hypertension. In yet another embodiment, the abnormal physiological state is type 2 overt or prediabetes mellitus. In a further embodiment, salt-sensitive essential hypertension is distinguished from salt-resistant essential hypertension. In yet a further embodiment, type 2 diabetes mellitus associated with the MgBD is distinguished from solely lipotoxic (lipid-induced) type 2 diabetes mellitus which is not associated with the MgBD.

In another aspect of the present invention, a binding pair member having high specificity for one or more of the peptides of the invention is provided. In one embodiment of this aspect, the binding pair is an antibody.

In a further aspect of the present invention, a method is provided for detecting the magnesium binding defect in serum or other body fluids. In one embodiment, this is an immunochemical procedure, such as competitive or sandwich assay.

In another aspect of the present invention, a method is provided for generating somatic cells having plasma membranes with reduced levels of tightly bound magnesium ion.

In yet another aspect of the present invention, an in vitro method is provided for screening substances which can promote the binding of magnesium ions to the plasma membranes of somatic cells and thereby ameliorate or correct the magnesium binding defect.

In a further aspect of the present invention, a method is provided for correcting the magnesium binding defect by the administration of the peptides of the invention or their peptide mimetics.

Magnesium is second only to potassium as an intracellular cation in man. It is an important regulator of cellular processes such as the formation and use of MgATP, the currency of metabolic energy of the cell. (Lehninger, 1975). This complex is required for the syntheses of tissue constituents, growth, thermogenesis and motility. Magnesium is a cofactor for more than 300 enzyme systems including some 100 systems which either produce or use MgATP (Allen et al., 1995). Therefore, it is evident that a decrease in the concentration of intracellular magnesium would very likely produce widespread, multiple disturbances in the metabolism of the cell.

Nevertheless, genetic and environmental factors influencing magnesium requirements and its metabolism remain poorly understood. Reports have shown that serum levels of magnesium do not reflect adequately the intracellular magnesium concentrations and acute elevations of the plasma magnesium concentration by the ingestion or infusion of magnesium salts rarely alter the magnesium concentration within the erythrocytes. It has also been reported that dietary supplementation with magnesium in untreated type 2 prediabetic patients does not influence intracellular magnesium concentrations, but did increase intracellular magnesium concentrations and total magnesium in plasma of type 1 diabetes mellitus patients (Corica et al., 1996; deValk et al., 1998; Eriksson and Kohvakka, 1995; Eibl et al., 1995). Gross depletion of tissue magnesium has been reported to be accompanied by a normal circulatory level of the cation (Wallach et al., 1962). Furthermore, reports show that there is little or no exchange of magnesium between the blood plasma and the erythrocytes, even though there is a 2:1 concentration gradient between the erythrocytes and the plasma (Searcy, 1961).

Salt-sensitive Essential Hypertension. The magnesium contents of the several anatomical compartments of whole blood from essential hypertension patients and normotensive control subjects have been quantified and several differences reported. The most noteworthy difference reported was the decreased levels of magnesium tightly bound to the erythrocyte plasma membranes of the hypertensive patients. The magnitude of the reduction in tightly bound magnesium correlated positively with the magnitude of the decreased concentration of intracellular magnesium, which in turn correlated negatively with the average blood pressures of the patients. It was also observed that this magnesium binding defect can be corrected by incubating the erythrocytes from essential hypertensive patients with blood plasma from the normotensive control subjects (Mattingly et al., 1991; U.S. Pat. Nos. 6,372,440 and 6,455,734).

The above investigations were extended to include two strains of genetic, salt-sensitive, hypertensive rats, SHR and SS/Jr rats, and their respective normotensive controls, WKY and SR/Jr rats (Wells and Agrawal, 1992). The magnesium binding defect was observed to occur in both strains of hypertensive rats as well as in the SR/Jr normotensive strain. It was concluded from these observations that the magnesium binding defect could only be a contributory, though perhaps a critical, cause of hypertension generation. Other investigators have collected evidence which indicates that the enzyme systems required for the extrusion of excess sodium ion from the cells, i.e. the Na⁺, K⁺-ATPase and/or the Na⁺, K⁺-cotransport enzyme, are defective in these two hypertensive rat strains. The presence of the MgBD indicates the decreased concentration of MgATP and therefore the decreased activity of enzymes which use MgATP, such as, Na⁺/K⁺-ATPase. The SR/Jr rat has the MgBD and is diabetic, but not hypertensive because the animal can increase the concentration of Na⁺/K⁺-ATPase (Rayson, 1988). Thus, the presence of the MgBD is a risk factor for salt-sensitive hypertension, but not the sole cause (Rayson, 1988). It has also been reported that the passive permeability of the cell membranes for sodium ion of the SHR rat may be greater than that of the control WKY rat.

The investigations, reported in further detail herein support several hypotheses about the mechanisms of hypertension generation in the SHR and SS/Jr rats. Without being bound by any theory of action, it is possible that, first, the magnesium binding defect in the cellular membrane of the vascular smooth muscle cell, for example, and perhaps those of all somatic cells, may permit, per unit of time, more than the normal amounts of sodium ion to enter passively into the cell even though the extracellular concentration of this ion is normal. Second, because the enzyme systems which remove excess sodium ion from the cell are defective, the intracellular sodium ion concentration increases to above normal levels. Third, because the extracellular sodium ion concentration tends to remain greater than the intracellular concentration, the sodium-calcium exchange enzyme within the cell membrane begins to export sodium ion from the cell and to import calcium ion. Fourth, the resulting increased intracellular calcium ion concentration stimulates the smooth muscle to contract. When the vascular smooth muscle contracts, the lumens of the arterioles in the peripheral circulation decrease in diameter thereby increasing the resistance to blood flow. Finally to overcome this increased resistance to blood flow, the heart will contract more strongly and this increased force is reflected as increased blood pressure. As noted above, the normotensive SR/Jr rat also has the magnesium binding defect. Nevertheless, this rat strain remains normotensive and can tolerate greatly elevated levels of dietary NaCl, ostensibly because its sodium ion extrusion enzymes increase and adequately prevent an increase in the intracellular concentration of this ion (Rayson, 1988).

Previous experiments employing the two salt-sensitive hypertensive rat strains established that the total intracellular concentrations of sodium and calcium are elevated, and the concentration of potassium is lower in the salt-sensitive, hypertensive SHR and SS/Jr rats, as compared to those of the normotensive WKY and SR/Jr rats (Wells and Blotcky, 2001; U.S. Pat. No. 6,372,440). This is entirely consistent with the above postulated mechanism of action.

It can be concluded from previous reports that: a) the MgBD is an important contributor to the causation of salt-sensitive hypertension, b) a normal component(s) of blood plasma is a necessary promoter of the binding of magnesium ion to the plasma membranes of cells, and c) the concentration of magnesium ion tightly bound to the plasma membrane controls the entrance of magnesium ion into the cell and consequently the variable concentrations of intracellular free and complexed magnesium ions since the concentration of intracellular bound magnesium is constant.

The discovery reported herein shows that the substances in normal human and rat plasmas which can ameliorate or correct the magnesium binding defect in erythrocyte membranes are the pentapeptide, Phe-Phe-Gly-Leu-Met-NH₂ (SEQ ID NO:1) and the tetrapeptide, Phe-Gly-Leu-Met-NH₂ (SEQ ID NO:2) (Wells and Agrawal, In press; U.S. Pat. No. 6,372,440). Both of these peptides occur at the C-terminal end of the tachykinin Substance P which has the amino acid sequence Arg-Pro-Lys-Pro-Gln-Gln-Phe-Phe-Gly-Leu-Met-NH₂ (SEQ ID NO:3). Evidence has been obtained to indicate that the generalized C-terminal sequence of the tachykinins (SEQ ID NO:4) embodies the substances in normal plasma which prevent the magnesium binding defect in cellular membranes. Furthermore, it has been discovered that the intravenous administration of the tetrapeptide of SEQ ID NO:2 to the salt-sensitive SS/Jr rat not only corrects the magnesium binding defect in erythrocytes of the SS/Jr rat, but also reduced its systolic blood pressure from an elevated value of 210 mm Hg to the control value, which is the blood pressure of the SR/Jr rats (Wells and Agrawal, In press). Thus, the correlation between the levels of the peptides of the present invention in body fluids and abnormal physiological states associated with MgBD is established by the discoveries reported herein.

Previous investigators have reported that, when injected intravenously, Substance P interacts with the baroreceptors on the surface of blood vessel endothelial cells causing vasodilation and reducing blood pressure. The effects of intravenously injected Substance P were observed with normotensive subjects, and the reduction in blood pressure resulted in lower than normal values (Qu and Stuesse, 1990). It has been unexpectedly discovered that the administration of the pentapeptide (SEQ ID NO:1) and/or the tetrapeptide (SEQ ID NO:2), each of which may be derived from Substance P by the action of amino-peptidases, to normotensive Sprague-Dawley rats had no effect on systolic blood pressure (Example 2). Furthermore, administration of the peptides of the present invention to hypertensive rats reduced blood pressure only to normal levels, not to below normal levels. Thus, the discovery that administration of the amidated peptides of the present invention reduce blood pressure was unexpected in view of reports by Qu and Stuesse (1990) that the effect of Substance P was to increase blood pressure to above normal levels. Additionally, the results reported herein unexpectedly revealed that the relatively stable pentapeptide (SEQ ID NO:1) and/or its contained tetrapeptide (SEQ ID NO:2) are several fold more active than Substance P in correcting the MgBD (Example 7).

In summary, in view of the previous reports for Substance P, it has been unexpectedly discovered, as described in further detail herein, that the pentapeptide (SEQ ID NO:1) and tetrapeptide (SEQ ID NO:2) are not active in reducing blood pressure of normotensive controls. Furthermore, as reported herein, the pentapeptide (SEQ ID NO:1) and tetrapeptide (SEQ ID NO:2) are significantly more active than Substance P in correcting the magnesium binding defect. While not wanting to be bound by any particular theory of action, the discoveries reported herein indicate that the molecular configuration of the pentapeptide (SEQ ID NO:1) and tetrapeptide (SEQ ID NO:2) are significantly different from that of Substance P. This may explain the reported differences in their effect on the magnesium binding defect and blood pressure in the experimental animals, i.e., the peptides are incorporated into the structure of the cell membrane, thus modifying its structure so as to permit the incorporation of Mg²⁺ ions and thus to increase the concentration of intracellular Mg and MgATP²⁻ ions.

It is apparent that an essential hypertensive person in whom the magnesium binding defect exists is, to a very high degree of probability, a salt-sensitive hypertensive and that the restriction of the dietary intake of sodium chloride (and other sources of sodium ion) by this individual would be therapeutically beneficial. In contrast, an essential hypertensive person without the magnesium binding defect is in all probability a salt-insensitive (Na⁺Cl-insensitive) hypertensive. Not only might such a person suffer needlessly if restricted to the minimum dietary sodium chloride intake consistent with a healthy existence but there is evidence to indicate that such a diet would be harmful for certain salt-insensitive essential hypertensive persons. The discovery reported herein of the substances in plasma which correct the magnesium binding defect makes possible a method for assessing a predisposition to salt-sensitive essential hypertension, and a method for distinguishing between these two forms of essential hypertension and makes possible the specific management of the disease.

It is further apparent that a method that would permit the rapid and accurate determination of the blood plasma levels of the peptides which have the amino acid sequences corresponding to those of SEQ ID NO:1, SEQ ID NO:2 and/or SEQ ID NO:4, all of which are active in preventing or correcting the magnesium binding defect, would be of value to a clinician for the assessment and treatment of essential hypertension. It is realistic to expect that when the blood plasma levels of these compounds are statistically significantly lower in an essential hypertensive patient, the magnesium binding defect is present and the hypertension can be classified as salt-sensitive. With the proper classification, appropriate treatment can be applied. On the other hand, if the concentrations of these substances are at least normal, then the magnesium binding defect is not present and the hypertension is classified as salt-insensitive.

Type 2 Diabetes Mellitus. It has been discovered that the magnesium binding defect occurs in the erythrocyte membranes of both normotensive and essential hypertensive, mildly affected type 2 diabetics. The characteristics of normotensive type 2 diabetes mellitus and control subjects and the values for the concentrations of the tightly bound magnesium ions in their erythrocyte membranes were determined and are described in further detail in Example 8. These discoveries make possible a method for assessing a predisposition to type 2 diabetes mellitus associated with MgBD, and a method for distinguishing between MgBD associated and solely lipid-induced type 2 diabetes mellitus.

While not wanting to be bound by any particular method of causation, the presence of the MgBD is believed to limit the amount of Mg²⁺ ion which enters the cell so that the intracellular concentrations of Mg²⁺ and MgATP²⁻ ions are significantly less than normal. These two functionally interdependent ions are required as cofactors and/or substrates for some three hundred enzyme systems involved in human metabolism. The decreased activities of particular ones of these enzymes systems due to the presence of the MgBD are responsible primarily for insulin resistance and, secondarily, for compensative hyperinsulinemia, the two pathophysiological processes embodied in type 2 prediabetes mellitus. These two processes can account for all of the morbid symptoms associated with this disease. Thus, the decreased intracellular ion concentrations, [Mg²⁺, MgATP²⁻], comprise the direct etiology of genetic type 2 prediabetes mellitus. These decreased intracellular ion concentrations can also result from the physiological, abnormal accumulation of saturated fatty acids in cell membranes (lipid-induced type 2 diabetes mellitus), and the result is increased insulin resistance. Overt type 2 diabetes mellitus is caused indirectly by the decreased intracellular [Mg²⁺, MgATP²⁻] and is present when insulin secretion is markedly reduced and hyperglycemia, rather than hyperinsulinemia, coexists with insulin resistance. The prevailing decreased insulinaemia in genetic overt type 2 diabetes results from the accumulation of amyloid in the islet cells; which in turn is the collateral effect on the pancreatic islet cells resulting from their production of the hyperinsulinemia of type 2 prediabetes. In the genesis of lipotoxic type 2 diabetes, the conversion of the prediabetic into the overt diabetic phase is due to enhanced saturated fatty acid accumulation in cell membranes of muscle and other somatic cells. This results in diabetes due predominantly to glucose toxicity and inhibition of insulin synthesis. Thus, if the MgBD is present, the individual has pre or overt genetic type 2 diabetes mellitus and is at risk for salt-sensitive essential hypertension. Individuals without the MgBD may develop lipotoxic type 2 diabetes mellitus, but are not at risk for salt-sensitive essential hypertension.

Preeclampsia/Eclampsia Syndrome. In addition to establishing the method for detecting the magnesium binding defect and the association of the defect with salt-sensitive essential hypertension and type 2 diabetes mellitus (U.S. Pat. Nos. 6,372,440 and 6,455,734), it has now been discovered, as reported herein (Example 4), that the occurrence of the magnesium binding defect is also associated with preeclampsia. Specifically, the evidence presented herein shows that the preeclampsia/eclampsia syndrome is associated with the occurrence of the magnesium binding defect in the plasma membranes of women who have presented the syndrome during pregnancy. These results suggest that the magnesium binding defect is at least a contributory cause of preeclampsia and provide a method to aid in the detection of a predisposition to presenting preeclampsia during pregnancy.

While not wanting to be bound by any particular method of causation, the results reported herein provide circumstantial evidence that the individual experiencing preeclampsia is in the prediabetic phase of type 2 diabetes mellitus, the stage prior to overt type 2 diabetes. This indicates that commonly experienced preeclampsia results from the imposition of pregnancy on type 2 prediabetes mellitus. Further, type 2 prediabetes is believed to be caused either by the presence of the MgBD, which is genetic, or to result from the accumulation of saturated free fatty acids on the cell membrane, sometimes referred to as lipid induced type 2 diabetes mellitus. Thus, these causes of type 2 pre or overt diabetes mellitus could be distinguished by measuring the concentration in body fluids of promoters of the magnesium ion binding to the plasma membranes of mammalian somatic cells.

The discoveries reported herein of the association between the MgBD and preeclampsia, and the amelioration of the MgBD with administration of the peptides of the invention makes possible a method to detect the presence of the magnesium binding defect and thereby assess a predisposition to preeclampsia. The discoveries reported herein are also useful in development of a therapeutic approach for the prevention or amelioration of the syndrome. Knowledge of the presence of the magnesium binding defect in the nulliparous patient would alert a physician to modify prenatal care according to conventional methods or procedures known in the art to minimize the effect of preeclampsia.

Detection of Peptide Levels in Blood Serum or Plasma. A preferred embodiment of the method of the present invention employs immunochemical procedures, such as competitive and sandwich assays (“binding assays”) to detect the occurrence of the magnesium binding defect. A variety of immunoassay methods are known in the art. See, e.g., Harlow and Lane, 1988, or Goding, 1986. Competitive and sandwich assays are well known and any competitive or sandwich assay may be used to practice the present invention, provided the benefits can be achieved. Exemplary sandwich assays are described in U.S. Pat. Nos. 4,376,110 and 4,486,530. Preferably, this invention involves binding assays wherein a binding pair member having affinity to one or more of the identified peptides is employed to detect the level of the peptide(s) in blood plasma or serum. A preferred binding pair member is an antibody. The exquisite specificity of antigen-antibody interactions has led to the development of a variety of immunologic assays. These assays can be used to detect the presence of either antibody or antigen and have played vital roles in diagnosing diseases and identifying molecules of biological and medical interest. These assays differ in their speed and their sensitivity; some are strictly qualitative, and others are quantitative. A strong antigen-antibody interaction depends on a very close fit between the antigen and antibody, which is reflected in the high degree of specificity characteristic of antigen-antibody interaction.

The strengths of the sum total of noncovalent interactions between a single antigen binding site on an antibody and a single epitope comprises the affinity of the antibody for that epitope. Low affinity antibodies bind antigen weakly and tend to dissociate readily, whereas high affinity antibodies bind antigen more tightly and remain bound longer. The association between a binding site on an antibody (Ab) with a monovalent antigen (Ag) can be described by the equation Ag+Ab_{←k-1}^{k}^{₁}^{→}Ab−Ag where k₁ is the forward (association) rate constant and k₋₁ is the reverse (dissociation) rate constant. The ratio of k₁/k₋₁ is the association constant K, a measure of affinity. The association constant K can be calculated from a ratio of the concentration of bound antibody-antigen complex to the concentrations of unbound antigen and antibody. K values vary for different antigen-antibody complexes and depend upon both k₁, which is expressed in liters/mole/second (L/mol/s) and k₋₁, which is expressed as L/sec. For polyclonal antibody preparations, K is not a constant because polyclonal antibodies are heterogeneous and in generally have a range of affinities. It is possible, using a means known in the art, such as a Scatchard plot, to determine the average affinity constant, Kₒ, by determining the value of K when half of the antigen binding sites are filled.

Any antibodies that have sufficiently high affinity for the target analyte may be used in the practice of the present invention, and preferably the antibodies are monoclonal antibodies. See, e.g., Harlow and Lane (1988), or Goding (1986). Affinity constants can be determined in accordance with any appropriate method known in the art, such as that described in Holvoet et al. (1994)(U.S. Pat. No. 6,309,888) which is incorporated in its entirety herein, by this reference. Although antibody-antigen reactions are highly specific, in some cases antibody elicited by one antigen can cross-react with another antigen. For example, such cross-reactions occur if two different antigens share an identical epitope. However, the cross-reacting antibody's affinity for one antigen may be considerably less than its affinity for the other antigen (Kuby, 1991), or the affinity of an antigen for a cross-reacting antibody may be below the detection limit of a given assay. This may result when, for example, an antibody is directed against a conformational epitope which is only efficiently exposed by one of the cross-reacting antigens. Monoclonal antibodies may be screened by any method known in the art, such as ELISA, IRMA and IEMA, and tested for specific immunoreactivity with the peptides of the invention or fragments thereof (Harlow and Lane, 1988).

Hybridomas from nonspecific B-cells can be selected out by means known in the art, such as mini-electrofusion described in Steenbakkers (U.S. Pat. No. 6,392,020). The specificity of the Mabs can be further evaluated by methods such as immunoprecipitation and T-cell agglutination tests as well as Western or immunoblots of polyacrylamide gels (U.S. Pat. No. 6,392,020). Techniques for raising and purifying antibodies are well known in the art, and any such techniques may be chosen to achieve the invention.

Both direct and indirect immunochemical procedures are used with known, or unknown but constant, concentrations of peptide (analyte) and antibody to determine the quantitative relationship between the two substances. This calibration procedure requires that either the analyte or antibody be labeled, either before or subsequent to binding, with an easily and accurately quantifiable material so that from the quantity of label present after the binding procedure and the previously determined binding relationship, the amount of analyte initially subjected to the binding reaction can be determined with acceptable accuracy. This methodology is widely known and extensively utilized because the extreme sensitivity possible with this method allows the quantification of physiological important, low molecular weight analytes such as steroid hormones, e.g. progesterone, in biological fluids, e.g. blood plasma, at concentrations in the picogram, i.e. 10-12 grams, per mL range. The construction and utilization of various such assay systems can be accomplished by one skilled in the art. Examples of such systems are described and discussed in detail in Chard (1990).

For the purpose of illustration, the construction of a suitable assay system for the determination of the concentrations of the peptides of interest in blood plasma is as follows. Since each of the peptides described herein has only one, and the same, immunological combining site, namely the pentapeptide of SEQ ID NO:1, suitable modifications of it will be used for labeling with a label such as the radioisotope, iodine-125, and for the raising of the necessary antibody. SEQ ID NO:1, its analog in which one of the phenylalanine (Phe) residues is replaced with a tyrosine (Tyr) residue, and its deamidated product are available from commercial sources, e.g. (Sigma Chemical Co., St. Louis, Mo.). The Tyr analog is labeled with iodine-125 by procedures described in the above reference and is used as the “trace analyte”. The deamidated peptide is conjugated with a carrier protein, as described below for use in the production in an animal of a polyclonal antibody having a high titer against the peptide.

Briefly, a polyclonal antibody is prepared by immunizing an animal with an immunogenic protein or peptide, such as SEQ ID Nos: 1, 2 and 4, and collecting antisera from that immunized animal. A wide range of animal species is used for the production of antisera, and the choice is based on the phylogenetic relationship to the antigen. Typically the animal used for production of antisera is a rabbit, a guinea pig, a chicken, a goat, or a sheep. Because of the relatively large blood volumes of sheep and goats, these animals are preferred choices for production of large amounts of polyclonal antibodies.

As is well known in the art, antigenic substances may vary in their abilities to generate an immune response. It is necessary in this case, therefore, to boost the host immune system by coupling such weak immunogens (e.g., a peptide or polypeptide) to a carrier, which is recommended in the present case. Examples of common carriers are keyhole limpet hemocyanin (“KLH”, which is preferred in this case) and bovine serum albumin (BSA). Means for conjugating a peptide or polypeptide to a carrier protein are well known in the art and include the use of MBS (m-malecimidobenzoyl-N-hydroxysuccimide ester), EDAC (1-ethyl-3-(3-dimethylaminopropyl) carbodiimide hydrochloride), and bisdiazotized benzidine. The conjugation and antibody production services are also available commercially (e.g., from Rockland, Immunochemicals for Research, Gilbertsville, Pa.). The pentapeptide SEQ ID NO:1 is used as the analyte standard.

As is also well known in the art, the immunogenicity of a particular immunogen can be enhanced by the use of non-specific stimulators of the immune response, known as adjuvants. Cytokines, toxins or synthetic compositions may also be used as adjuvants. The most commonly used adjuvants include Freund's complete adjuvant (a non-specific stimulator of the immune response containing killed Mycobacterium tuberculosis) and incomplete Freund's adjuvant which does not contain the bacteria.

Milligram quantities of antigen (immunogen) are preferred although the amount of antigen administered to produce polyclonal antibodies varies with the nature and composition of the immunogen as well as with the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various times following inoculation.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate monoclonal antibodies (MAbs).

For production of rabbit polyclonal antibodies, the animal can be bled through an ear vein or alternatively by cardiac puncture. The removed blood is allowed to clot and then centrifuged to separate serum components from whole cells and blood clots. Sterility is maintained throughout this preparation. The serum may be used as such for various applications or else the desired antibody fraction may be isolated and purified by well-known methods, such as affinity chromatography using another antibody, a peptide bound to a solid matrix, or by using procedures such as, protein A or protein G chromatography.

Instead of using polyclonal antibodies, monoclonal antibodies (MAbs) are preferably used in the practice of this invention. MAbs may be readily prepared through use of well-known techniques, such as those exemplified in U.S. Pat. No. 4,196,265, incorporated herein by reference. Typically, this technique involves immunizing a suitable animal with a selected immunogen, e.g., a purified or partially purified protein, polypeptide, peptide or domain. The immunizing substance is administered in a manner effective to stimulate antibody producing cells.

The methods for generating monoclonal antibodies (MAbs) generally begin along the same lines as those for preparing polyclonal antibodies. Monoclonal antibodies with affinities of 10⁻⁸M⁻¹ or preferably 10⁻⁹ to 10⁻¹⁰M⁻¹ or stronger will typically be made by standard procedures as described, e.g., in Harlow and Lane, 1988 or Goding, 1986. Rodents such as mice and rats are preferred animals; however, the use of rabbit, sheep, or frog cells is also possible. The use of rats may provide certain advantages (Goding, 1986), but mice are preferred, with the BALB/c mouse being most preferred as it is routinely used and generally gives a higher percentage of stable fusions.

The animals are injected with antigen, generally as described above. The antigen may be coupled to carrier molecules such as keyhole limpet hemocyanin if necessary. The antigen is typically mixed with adjuvant, such as Freund's complete or incomplete adjuvant. Booster injections with the same antigen are made at approximately two-week intervals.

Following immunization, somatic cells with the potential for producing antibodies, specifically B lymphocytes (B cells), are selected for use in the MAb generating protocol. Antibody-producing B cells are usually obtained by disbursement of the spleen, but tonsil, lymph nodes, or peripheral blood may also be used. Spleen cells are preferred because they are a rich source of antibody-producing cells that are in the dividing, plasmablast stage.

The antibody-producing B lymphocytes from the immunized animal are then fused with cells of an immortal myeloma cell line, generally one from the same species as the animal that was immunized. Myeloma cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas). Any one of a number of myeloma cells may be used, as is known to those of skill in the art (Goding, 1986).

Methods for generating hybrids of antibody-producing spleen or lymph node cells and myeloma cells usually comprise mixing somatic cells with myeloma cells in about a 2:1 proportion in the presence of an agent or agents (chemical or electrical) that promote the fusion of cell membranes. The original fusion method using Sendai virus has largely been replaced by those using polyethylene glycol (PEG), such as 37% (v/v) PEG, as has been described in the art. The use of electrically induced fusion methods is also appropriate.

Fusion procedures usually produce viable hybrids at low frequencies. However, this does not pose a problem, as the viable, fused hybrids are differentiated from the parental, unfused cells (particularly the unfused myeloma cells that would normally continue to divide indefinitely) by culturing in a selective growth medium. The selective medium is generally one that contains an agent that blocks the de novo synthesis of nucleotides. Exemplary and preferred agents are aminopterin, methotrexate, and azaserine. Aminopterin and methotrexate block de novo synthesis of both purine and pyrimidine nucleotides, whereas azaserine blocks only de novo nucleotide purine synthesis. Where aminopterin or methotrexate is used, the media is supplemented with hypoxanthine and thymidine as a source of nucleotides (HAT medium) by salvage pathways. Where azaserine is used, the media is supplemented with hypoxanthine.

A preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and therefore, they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which particular clones are selected. The selection of hybridomas is performed by culturing the cells in microtiter plates, followed by testing the individual clonal supernatants (after about two to three weeks) for antibody producers using ELISA IgG assays. Antibody positive hybridomas are screened further for MAbs with desired reactivity using antigen based assays. Such assays are normally sensitive, simple, and rapid, such as radioimmunoassays, enzyme immunoassays, dot immunobinding assays, and the like.

The selected hybridomas are then serially diluted and cloned into individual antibody-producing cell lines, clones of which are then propagated indefinitely to provide MAbs. The cell lines can be exploited for MAb production in two basic ways.

A sample of the hybridoma can be injected often into the peritoneal cavity of a histo-compatible animal of the type that was used to provide the somatic and myeloma cells for the original fusion, such as a syngenetic mouse. Optionally, the animals are primed with a hydrocarbon, especially oils such as pristane (tetramethylpentadecane) prior to injection. The injected animal develops tumors secreting the specific monoclonal antibody produced by the antibody producing hybridoma. The ascites fluid of the animal, and in some cases blood, can then be obtained to provide MAbs in high concentration.

The individual cell lines could also be cultured in vitro; where the MAbs are naturally secreted into the culture medium from which they can be readily obtained in high concentrations. MAbs produced by either means may be further purified, if desired, using filtration, centrifugation and various chromatographic methods such as HPLC or affinity chromatography. Monoclonal antibodies are preferred since the hybridoma cells which produce them can be kept in vitro indefinitely, and the assay can be more accurate due to the higher selectivity that can be achieved with a polyclonal antibody assay. The raising of monoclonal antibodies is well known. Means for preparing and characterizing antibodies are also well known in the art (Harlow and Lane, 1988).

Whether a monoclonal or polyclonal antibody is employed in the practice of this invention, the steps involved in carrying out an assay consistent with the teachings of this invention are the same. Broadly speaking, the assay of this invention involves first constructing a standard curve involving known concentrations and amounts of reagents that subsequently can be used in assays where the concentration of the polypeptide or peptide associated with the magnesium binding defect is being determined in plasma or other body fluid samples. These techniques, while not previously practiced in connection with these specific peptides, are otherwise known in the art as having been practiced in the detection of other analytes.

For purposes of example, in the context of this invention, an assay can be practiced as follows. One way to construct an assay is to use the radioimmunoassay (“RIA”). As is known in the art, the principle of RIA involves competitive binding of radiolabelled antigen and unlabeled antigen to a high-affinity antibody. Standard curves are constructed from data gathered from a series of samples each containing the same known concentration of labeled antigen, and various, but known, concentrations of unlabeled antigen. Antigens are labeled with a radioactive isotope tracer. The mixture is incubated in contact with an antibody. Then the free antigen is separated from the antibody and the antigen bound thereto i.e., the antibody-antigen complex. Finally, by use of a suitable detector, such as a gamma or beta radiation detector, the percent of either the bound or free labeled antigen or both is determined. This procedure is repeated for a number of samples containing various known concentrations of unlabeled antigens and the results are plotted as a standard graph. The percentages of bound tracer antigens are plotted as a function of the antigen concentration. Typically, as the total antigen concentration increases the relative amount of the tracer antigen bound to the antibody decreases. After the standard graph is prepared, it is thereafter used to determine the concentration of unlabeled antigen in samples undergoing analysis.

In an analysis, the sample in which the concentration of antigen is to be determined is mixed with a known amount of tracer antigen. Tracer antigen is the same antigen known to be in the sample but which has been labeled with a suitable radioactive isotope. The sample with tracer is then incubated in contact with the antibody. Then it can be counted in a suitable detector which counts the free antigen remaining in the sample. The antigen bound to the antibody or immunoadsorbent may also be similarly counted. Then, from the standard curve, the concentration of antigen in the original sample is determined.

The first step in a standard curve is to incubate a fixed amount of the tracer analyte with a reagent blank and with a series of dilutions of the antibody in constant volumes of buffer containing bovine serum albumin (“BSA”). At the end of the incubations each of the antibody-tracer analyte complexes formed are precipitated by the addition of constant amounts of polyethyleneglycol 4000. The level of radioactivity of each precipitate is determined with the use of a suitable gamma counter. These values, in descending order, are plotted on the ordinate (normal scale) of semilog paper against the dilutions of antibody, in descending order (highest to lowest), plotted on the abscissa (log scale).

From this plot is read the dilution of antibody which combines with 50 percent of the labeled analyte. This particular combination of concentrations of tracer analyte and antibody is used for the construction of the standard curve.

To construct the standard curve, a second incubation is carried out under the same conditions as before except that the above dilution of antibody and concentration of tracer analyte are introduced together into a series of incubation tubes containing increasing concentrations of the standard analyte. The processes of incubation, precipitation of the antibody-analyte complexes, and quantification of the radioactivities are carried out as before. This time, a plot on semilog paper of the levels of radioactivity in descending order on the ordinate against the concentrations of standard analyte in ascending order on the abscissa is prepared and constitutes in the standard curve.

To determine the quantity of the peptide in a test sample, a third incubation is carried out using the conditions used in constructing the standard curve except that the ascending concentrations of standard analyte are replaced by two or more suitable dilutions of a concentrate of plasma peptides prepared as indicated above. The concentrations of analyte added to the incubation tubes are read from the standard curve by noting the concentration of standard that corresponds to each level of radioactivity measured for the tubes containing the unknowns. Such procedures can, of course, be automated, as is known in the art. From the standpoint of good practice the standard curve should be developed each time unknown samples are assayed.

When the assay procedure of appropriate sensitivity and specificity has been constructed, it is calibrated by comparing the concentrations, i.e. nanograms per milliliter, of the analyte found in a series of samples of normal human blood plasma with the directly determined values of magnesium bound in the erythrocyte membranes from the same donors. The method for the direct determination of bound magnesium in erythrocyte membranes is described in detail in Mattingly et al. (1991) and Example 1 below. If the relationships between the members of these two sets of values are constant within experimental error, the average of the plasma concentrations of peptides of interest is considered to be the equivalent of the average value of normal erythrocyte membrane-bound magnesium. Therefore, a value of plasma peptide determined for patients which is significantly less than the average of those determined normal values, is considered to indicate the presence of the magnesium binding defect in the somatic cells of that patient.

Once the conditions for the standard curve have been fine-tuned or adjusted so that the curve includes the concentrations of analyte likely to be encountered in a specific biological fluid, e.g. blood plasma, the standards and reagents, together with appropriate instructions for use, can be packaged in a “kit” for commercial distribution.

The principles involved in the radioimmunoassay system above can also be applied to a variety of immunoassay systems, preferably competitive binding assays, of varying degrees of sensitivity for the qualification and quantification of the peptides involved in the detection of the magnesium binding defect. In each case, the antibody can be monoclonal or polyclonal. It is bound to a support so that the antibody-analyte complexes can be readily separated from the incubation mixtures. The labels used for forming the tracer analyte determine the sensitivities of the systems and provide for colorimetric (least sensitive), radioactive, fluorometric and chemiluminescent (most sensitive) endpoints. Consequently, the conventional apparatuses used for the determination of each type of endpoint will be required.

There are many ways the antibody may be bound and the tracer analyte labeled. For example, the antibody can be bound in the following ways:

a) Antibody adsorbed on a polystyrene tube or surface (microtiter plate). Complexes are isolated by washing.

b) Antibody adsorbed on a polyvinyl tube or surface (microtiter plate). Complexes are isolated by washing.

c) Antibody adsorbed on 6 mm polystyrene spheres. Complexes are isolated by centrifugation and washing.

d) Antibody adsorbed on 6 mm polyvinyl spheres. Complexes are isolated by centrifugation and washing.

e) Antibody bound to 5 urn microparticles of paramagnetic ferrous oxide. The surfaces of particles are derivitized with a substance that has a terminal amino group. The antibody is linked to the surface by the use of glutaraldehyde. Complexes are isolated by applying a magnetic field to hold the particles against the surface of the incubation tube, and then washing.

f) Antibody bound to 5 um microparticles of paramagnetic chromium dioxide. Binding of antibody to surface of particles and isolation of complexes accomplished as described in (e) above.

g) Antibody bound to sepharose (an insoluble complex carbohydrate) after the surface of the sepharose is modified by use of cyanogen bromide. Complexes are isolated by centrifuging and washing.

h) Antibody bound to agarose (an insoluble complex carbohydrate). Binding of antibody and isolation of complexes are accomplished as in (g) above.

i) Antibody derivitized with biotin by use of biocytin and glutaraldehyde. (Biotinyl-N-hydroxysuccimide ester, or biotinyl-p-nitrophenyl ester, or caproylamidobiotinyl-N-hydroxy-succimimide ester may also be used for biotinylation). Complexes are isolated by allowing complex to combine with the protein avidin which is bound to a solid support such as plastic spheres, paramagnetic particles, or insoluble carbohydrates by the methods indicated above.

In general, the detection of immunocomplex formation is well known in the art and may be achieved through the application of a number of tags besides the radioactive tag used in the RIA described above. These other methods are based upon the detection of fluorescent, biological, or enzymatic tags, for example. Some examples include the following:

1) Analyte conjugated with horseradish peroxidase using glutaraldehyde. Quantification is accomplished by: a) measuring intensity of color produced in the presence of hydrogen peroxide and o-phenylenediamine or preferably 3,3′,5,5′-tetramethylbenzidine; b) measuring fluorescence intensity after the addition of hydrogen peroxide and fluorescein or rhodamine; c) by measuring chemiluminescence intensity after the addition of hydrogen peroxide and luminol plus benzothiazole. The equipment required includes colorimeter or spectrophotometer (unaided normal vision sufficient for qualitative assessment), spectrofluorimeter, or luminometer, respectively.

2) Analyte labeled with acridinium ester by use of 4-(2-succinimidyloxy-carbonyl ethyl)-phenyl-10-methylacridinium-9-carboxylate fluorosulfonate. Quantification is accomplished by measuring intensity of chemiluminescence produced by the addition of alkaline hydrogen peroxide. A luminometer is required.

3) Analyte labeled with fluorescein isothiocyanate. Quantification is accomplished by measuring the intensity of fluorescence after addition of hydrogen peroxide and a peroxidase such as horseradish peroxidase. A spectrofluorimeter is required.

4) Analyte labeled with alkaline phosphatase by use of glutaraldehyde. Quantification is accomplished by measuring intensity of fluorescence after addition of 4-methylumbelliferylphosphate. A spectrofluorimeter is obviously required.

5) Analyte labeled with rhodamine isothiocyanate. Quantification is accomplished the same as in (3) above.

6) Analyte labeled with glucose-6-phosphate dehydrogenase by use of glutaraldehyde. Quantification is accomplished by measuring ultraviolet light absorbed after the addition of glucose-6-phosphate and nicotinamide-adenine dinucleotide. A UV spectrophotometer is required.

7) Analyte labeled by conjugation with bacterial peroxidase. Conjugation and quantification is accomplished the same as in (1) above.

By use of one of the various bound forms of the antibody and of labeled forms of analyte described above it is possible to construct many kinds of competitive binding assay systems for the quantification of the pentapeptide (SEQ ID NO:1) and its tetrapeptide degradation product (SEQ ID NO:2) which occur in human blood plasma and prevent the occurrence of the magnesium binding defect in cell membranes. Alternatively, in each such system, the tracer analyte, rather than the antibody, can be the bound member of the system. Since these systems vary in their sensitivities and equipment requirements, it is possible to select a system according to the specific requirements of or adapted to the existing equipment employed by the user.

The specific reagents and other requirements for each system, except hardware, and directions for use can be packaged in “kits” containing various combinations of the above reagents, together with the necessary containers containing washing solutions, for commercial distribution.

Screening for Promoters of Magnesium Binding. Another aspect of the present invention provides an in vitro system for identifying substances which promote the binding of magnesium ions to the plasma membranes of somatic cells (tightly bound magnesium). A critical discovery for this aspect was the generation of a deficit of membrane bound magnesium ions in normal erythrocytes or other somatic cells (“deficient cells”). Briefly, deficient cells were generated by allowing normal cells to age in a cell stabilizing buffer including about 1 to 1.5 mg/ml of sodium deoxycholate, at about 4° C. While Alsever's solution, including 1.25 mg/ml sodium deoxycholate is preferred, other cell stabilizing buffers may be used as those of skill in the art would recognize. Identification of substances which promote binding of magnesium ions can then be performed by incubating the deficient cells at about 37° C. in a physiological medium which includes magnesium ion, such as Krebs Ringer phosphate glucose plus magnesium ion (Dawson et al., 1962), to which is added the potential binding promoter. For example, the C-terminal sequence of the tachykinins (SEQ ID NO:4), or the pentapeptide (SEQ ID NO:1) and its contained tetrapeptide (SEQ ID NO:2) which occur in normal blood plasma and which may be derived from the C-terminal sequence of tachykinins, such as Substance P, by the action of plasma aminopeptidases, may be used in this method to promote binding of magnesium.

The results of this method were confirmed in vivo by the intravenous administration of 3.0 μgm/kg of the tetrapeptide (SEC ID NO:2) to 300 gm SS/Jr salt-sensitive, hypertensive rats and Sprague-Dawley normotensive, control rats. Within 10 minutes the blood pressures of the hypertensive animals had fallen to control values and the MgBD in their erythrocytes was corrected while these values in the control rats were unaffected. These result provide evidence that the occurrence of hypertension in the SS/Jr rats is dependent on the presence of the MgBD in the plasma membranes of the smooth muscle cells in the blood vessels. Further, it is reasonable to conclude that, in general, the occurrence of any pathophysiological state which depends on the presence of the MgBD will be affected by the amelioration or correction of the MgBD and the consequent decreased intracellular concentrations of free and complexed magnesium ions.

The stringent interdependence of the intracellular concentrations of free magnesium ion, i.e., [Mg²⁺], and of its complex with ATP, i.e., [MgATP²⁻], is evident from consideration of the dissociation of the complex. The mathematical expression of the dissociation constant, K_{diss}, for the dissociation, i.e., MgATP²⁻ [symbol] Mg²⁺+ATP⁴⁻, of the complex which is ([ATP⁴⁻])×([Mg²⁺]÷[MgATP²⁻])=1×10⁻⁵. The symbol [Mg²⁺] represents the molar concentration of the free magnesium ion in the intracellular compartment containing the ATP⁴⁻ etc. From this equation it is clearly evident that the value of the [ATP⁴⁻] in a particular cell determines the value of the ratio of the [Mg²⁺] to [MgATP²⁻] in that cell. Thus, since in the cell the [ATP⁴⁻] is involved in several equilibria, e.g., a critically important one of which is the hydrogen ion concentration, which is normally maintained constant, the [ATP⁴⁻] would also be maintained constant by metabolic processes. Thus, as a consequence, the value of [Mg²⁺]: [MgATP²⁻] is also constant. Therefore, a change in the concentration of either member of this ratio requires that the concentration of the other member change proportionately. Since the ratios remain constant, the cell can appear to remain normal even when the decreased concentration of the two ions have greatly altered cell metabolism.

There is an additional consideration in the above discussion concerning salt-sensitive, essential hypertension. The evidence presented herein indicates that the MgBD occurs uniformly in all of these individuals and they become hypertensive because of decreased activities of sodium extrusion enzymes such as Na⁺/K⁺-ATPase. However, not all individuals with the MgBD do become hypertensive. This was the case with the type 2 diabetes mellitus patients involved in the present study who were normotensive but had the defect, while other individuals with the defect were observed to be hypertensive (Mattingly et al., 1991). This situation is also evident in the SS/Jr and SR/Jr rats; both have the MgBD but only the former is hypertensive (Wells and Agrawal, 1992).

An explanation for this dichotomy has been proposed (Rayson, 1988). Rayson obtained evidence for the existence of a “homeostatic response” which does not occur, or is defective, in SS/Jr rats but does occur in the salt-resistant, i.e., the SR/Jr rat. When the “homeostatic response” is normal, the animal can synthesize additional amounts of sodium ion extrusion enzymes, such as Na⁺/K⁺-ATPase, which in the presence of decreased intracellular concentration of Mg²⁺ and MgATP²⁻ prevents the accumulation of intracellular ions and consequently the development of hypertension. Since the “homeostatic response” is in all likelihood genetic, the occurrence of the MgBD can only indicate the risk of development of salt-sensitive hypertension. However, the decreased intracellular [Mg²⁺] and [MgATP²⁻] resulting from the presence of the MGBD can account for all aspects of type 2 diabetes mellitus and, while not wanting to be bound by any particular mode of action, it is believed that the presence of this defect has a causative role in the development of this disease.

### EXAMPLES

The present invention is described by reference to the following Examples which are offered by way of illustration and are not intended to limit the invention in any manner. Standard techniques well known in the art or techniques specifically described below were utilized.

### Example 1

### Method for Measurement of Tightly Bound Magnesium in Plasma Membranes of Somatic Cells

Approximately 40 ml of heparinized venous blood were collected from human subjects at least three hours postprandially and placed immediately on ice. Magnesium tightly bound to the erythrocyte membrane was measured using the packed cells from approximately 5 ml of the heparinized venous blood samples. The cells were suspended in approximately 10 ml each of the following buffered (Tris+Tris.HCl, 5.0 mM, pH 7.4) solutions at 5° C.: twice in erythrocyte wash (sucrose, 280 mM), and three times in hemolyzing wash (sucrose, 14 mM). The final erythrocyte membranes that remained intact after hemolysis (ghosts) were each suspended in 0.15 M NaCl solution to a volume of 25.0 ml. Magnesium and protein concentrations of these suspensions were determined by atomic absorption spectrophotometry and the method of Bradford (1976) using the reagent obtained from Bio-Rad Laboratories (Richmond, Calif.), respectively. The Bradford method was standardized with bovine serum albumin (Sigma Chemical Co., St. Louis, Mo.).

Measurement of tightly bound magnesium was also performed with erythrocyte membranes from male rats (Harlan Sprague Dawley, Indianapolis, Ind.). Body weights (grams) were as follows: SHR. avg. 341, range 326-355; WKY, avg. 263, range 246-283; SS/Jr. avg. 455, range 420-500; SR/Jr. avg. 453, range 420-490. The SS/Jr and SR/Jr rats were retired breeders. To confirm that the appropriate animals were hypertensive, the systolic blood pressures were measured by recording from a tail cuff applied to the rat's tail, warmed for ten minutes with a heat lamp. The results were as follows (in mmHg): SHR, avg. 212, range 189-229; WKY not measured: SS/Jr. and SRYJr. one of each selected at random, 210 and 160, respectively.

Blood was collected in individual centrifuge tubes containing Na+-heparin, and immediately placed onto ice. The subsequent procedure used to isolate the erythrocyte membranes from intracellular contents was performed at 4° C. Ice-cold buffers were used and the procedure was designed to be compatible with the requirements of neutron activation analysis (NAA), i.e., as free as possible of Na+ and Cl⁻.

An 8 mL sample of blood from each animal was centrifuged (1500×g, 15 min) and the plasmas from the SHR and WKY rats were pooled separately and reserved for a repetition of the incubation experiments previously described (Mattingly et al., 1991; Wells and Agrawal, 1992). The erythrocytes were then suspended in 5.0 mL portions of 0.15 M LiCl, the suspensions centrifuged as above, and the supernatants discarded. Each cell residue was hemolyzed by suspending it in another 5.0 mL portion of 0.15 M LiCl and rapidly mixing the suspension into 100 mL of buffer containing 10 mM Tris-Tris formate plus 25 mM LiCl (pH 7.40). These clear mixtures were allowed to stand on ice for a few minutes. They were then centrifuged (28 000×g. 15 min), and the separate supernatants were retained. The residues were washed by suspending them in 50 mL of the Tris-LiCl buffer and centrifuging as before. The resulting supernatants were added to the corresponding retained supernatants. The residues were washed again, using 25 mL of the same buffer, and the supernatants were retained as before. Finally, the cell residues were washed twice as above with 25 mL portions of 10 mM Tris-Tris formate buffer (pH 7.40). These supernatants were also saved as above, thus each of the supernatant pools contained a minimum total volume of approximately 230 mL. Total volumes were recorded and the supernatants were stored at 4° C.

Between the last two washings of the cell residues, the penultimate cell residues were gently suspended by slowly pouring the 25 mL of fresh buffer onto the residue and carefully decanting the suspension of faintly red, erythrocytic membranes. This decanting procedure was repeated as necessary to remove all of the dark red residue.

A portion of each of the separate membrane residues were suspended in the 10.0 mM Tris-Tris formate buffer to a final volume of 25 mL. The protein contents of these suspensions were determined with the use of a Bicinchoninic acid protein assay kit and procedure (Sigma Chemical Co., St. Louis, Mo.). This method was standardized with the use of bovine serum albumin. The Mg²⁺ contents of these suspensions were determined by atomic absorption spectrometry (AAS; Varian, SpectraAA 200) after aliquots of the pooled supernatants were diluted appropriately with ultrapure water.

Determination of the protein contents of these suspensions of membranes permitted the concentrations of tightly bound Mg²⁺ in the erythrocytic membranes to be expressed as ug/2.0 mg of membrane protein. The sums of the protein contents of each related pair of the membrane suspensions were therefore the totals of the membrane proteins involved in the analyses of the several blood samples and consequently permitted the concentration of magnesium to be expressed relative to 2.0 mg of the membrane protein. The pooled supernatants resulting from the preparation of the erythrocyte membranes contained the intracellular contents and were analyzed for total Mg²⁺. Table 1 contains the results of the analyses of the erythrocytes from the two strains of genetic hypertensive rats examined and their customary, normotensive controls.

TABLE 1
Total Intracellular Cation Concentrations^{a} in Rat Erythrocytes.

| Analysis | | Rat strain | | | Rat strain | | |
|---|---|---|---|---|---|---|---|
| Cation | Method^{b} | SHR | WKY | P^{c} | SS/JR^{d} | SR/JR^{d} | P^{c} |
| Mg²⁺ | AAS | 7.75 ± 0.41 (8) | 11.251 ± 0.08 (7) | <0.01 | 7.28 ± 0.29 (7) | 7.40 ± 0.46 (8) | >0.8 |

^{a}Microgram of cation per 2.0 mg of erythrocyte membrane protein; mean ± SE; number of samples is in parentheses. Due to an unrecognized mathematical error in the measurement of erythrocyte membrane protein, a higher value was previously reported, i.e., ug of cation per 0.50 mg of protein. See Mattingly et al. (1991) and Wells and Agrawal (1992).
^{b}AAS, atomic absorption spectroscopy.
^{c}Student's t test.
^{d}Dahl-derived salt-sensitive (SS) and salt-resistant (SR) rats.

The concentrations of tightly-bound membrane Mg²⁺ are consistent with those observed in previous studies (Wells and Agrawal, 1992). They indicate that the MgBD occurs in both the SHR and SS/Jr rats, which are hypertensive and also possess defective enzyme systems for the extrusion of excess Na⁺ from their cells (Rosati et al., 1988; de Mondonca et al., 1985). The MgBD also occurs in the SR/Jr rat, the control for the SS/Jr rat, but does not occur in the WKY rat, the control for the SHR rat. However, both of these control strains adequately extrude excess Na⁺ from their cells (Rosati et al., 1988; de Mondonca et al., 1985). The concentrations of total intracellular Mg²⁺ among the four rat strains varied directly with the levels of membrane bound Mg²⁺ as noted previously-(Mattingly et al., 1991; Wells and Agrawal, 1992).

### Example 2

### In Vivo Effectiveness of the Tetrapeptide of SEQ ID NO:2 on Magnesium Binding and Blood Pressure

The effectiveness of the tetrapeptide (SEQ ID NO:2), which appears to be the most active in vitro as a magnesium binding promoter, was tested in vivo in 300-350 gm. male Sprague-Dawley normotensive (control) and in Dahl derived, salt-sensitive rats (SS/Jr rats). The intravenous administration of this substance to genetic hypertensive SS/Jr rats and normotensive Sprague-Dawley control rats corrected the magnesium binding defect and the hypertension in the former and had no influence on these parameters in the latter. In this procedure the systolic blood pressure was measured in conscious animals by using the photoelectric, tail-cuff, compression method as follows. The animal was placed in an incubator at 40° for twenty minutes in order to elevate its body temperature. The cuff, which was placed around the tail, included sensor which provided the pressure signal which was recorded onto a chart. After the pre-injection pressure was recorded, the animal was immediately injected through the tail vein with 168 uL of a freshly prepared solution of the tetrapeptide (SEQ ID NO:2) in normal saline.

In the initial experiments, the blood pressure was recorded at 5, 10, 30, 60 and 300 minutes after the administration of the tetrapeptide. There was no further decrease in the blood pressure after 10 minutes and the blood pressure returned to the pre-injection level within 60 minutes. Therefore, in further experiments, the blood pressure of each animal was recorded 10 minutes after the injection of the tetrapeptide. The results are displayed in Table 2.

The rats were allowed to rest for one day and then one-half of the SS/Jr and one-half of the Sprague-Dawley rats were injected as above with 3.0 ugm/kg of the tetrapeptide (SEQ ID NO:2). All of the rats were sacrificed by decapitation 30 minutes later and the blood from each rat was collected in a centrifuge tube containing sufficient heparin to prevent clotting. The blood samples were centrifuged (1,000×g, 20 minutes) at 5° C. and the individual plasma layers were removed and frozen. The residues of erythrocytes were suspended three times in cold, normal saline and then were hemolyzed by the addition of 15 mL of cold hemolyzing buffer (10 mM Tris Tris HCl, pH 7.4). This buffer was then used in the isolation of the erythrocyte membranes from these hemolysates and in the determination of their total magnesium and protein contents as described above. The concentrations of tightly bound magnesium in the erythrocyte membranes are shown in Table 2.

It can be seen from Table 2 that the average blood pressure of the injected SS/Jr rats was intermediate between that of the uninjected SS/Jr rats and the Sprague-Dawley rats. However, it was the same as the values reported for the salt-resistant SR/Jr rats, i.e., the controls for the SS/Jr rats. Thus the tetrapeptide reduced the hypertensive blood pressure of the SS/Jr rats to the normal blood pressure of their controls, and did not affect the blood pressures of the normotensive Sprague-Dawley rats.

The glucose concentrations in the reserved plasma samples were determined by the anthrone method (Rendina, 1971). The concentrations in the injected SS/Jr rats were not different from those of the SS/Jr rats which were not injected with the tetrapeptide. This was also the case with the Sprague-Dawley control rats. Thus, the values of the SS/Jr rats were combined and compared with the combined, control values of the Sprague-Dawley rats as follows: 149.2±7.5 (15) vs. 100.7±11.2 (4) mg/100 mL., P<0.01. Presumably, the dose of tetrapeptide (SEQ ID NO:2) (3.0 ugm/kg), administered and the time allowed for its effect to be evident were insufficient to affect the blood glucose levels of the SS/Jr rats.

TABLE 2
Effectiveness of Tetrapeptide In Vivo

| | | Magnesium | | Blood | |
|---|---|---|---|---|---|
| Rat strain | Injected* | Binding | P | Pressure | P |
| SS/Jr | No | 298 ± 16 (7) | <0.01 | 190 ± 2.7 (15) | <0.01 |
| SS/Jr | Yes | 415 ± 20 (8) | | 154 ± 3.3 (15) | |
| Sprague- | No | 405 ± 13 (4) | NS | 114 ± 6.2 (4) | NS |
| Dawley | | | | | |
| Sprague- | Yes | 414 ± 10 (4) | | 117 ± 6.8 (4) | |
| Dawley | | | | | |

*Injected I.V. with 168 μL of normal saline containing 1, 3 or 10 μgm of the tetrapeptide (SEQ ID NO: 2). The latter doses in SS/Jr and all doses in Sprague-Dawley rats had the same effect
**Nanograms of magnesium per 0.50 mg of membrane protein
⁺Millimeters of mercury

### Example 3

### Remediation of Magnesium Binding Defect with In Vitro Administration of Peptides

The effect of the tetrapeptide (SEQ ID NO:2) and pentapeptide (SEQ ID NO:1) on the binding of magnesium by the erythrocyte membrane was ascertained by measuring the magnesium contents of ghosts after the erythrocytes from essential hypertensive patients and normal controls were incubated with one or both of the peptides. For this purpose, two 10 ml samples of heparinized blood from essential hypertensive subjects were utilized. After the plasma was separated from the erythrocytes, the erythrocytes were washed twice with 10 ml of cold erythrocyte wash and the peptides were then added to the erythrocytes from the hypertensive subjects and the mixtures were incubated for 3 hrs. at 37° C., chilled on ice and the erythrocyte ghosts were prepared and analyzed for magnesium and protein as described in Example 1.

The results of studies in which erythrocytes were incubated with the peptides revealed that the decreased binding of magnesium to the erythrocyte membranes of the hypertensive subjects was returned to normal when such erythrocytes were incubated with the tetrapeptide and/or pentapeptide.

### Example 4

### Association of Magnesium Binding Defect with Preeclampsia Syndrome

The possible relationship between magnesium binding defect and preeclampsia was investigated by measuring tightly bound magnesium in erythrocyte membranes from six women who had presented preeclampsia during pregnancy and from six women who did not present preeclampsia during pregnancy. These women were not obese, were in good health, and were normotensive, and had been in their twenties and early thirties when pregnant.

Erythrocyte membranes were isolated from duplicate 5 ml. samples of heparinized venous blood as described in Example 1. The magnesium contents of these membranes were measured by atomic absorption spectrometry, and total protein content was measured by the Bicinchoninic Acid method (Sigma Chem. Co., St. Louis, Mo.), as described in Example 1. The results of these measurements revealed reduced binding of magnesium to the erythrocyte membranes only in women who presented with preeclampsia during pregnancy (Table 3). This discovery establishes involvement of the magnesium binding defect in the pathogenesis of preeclampsia.

TABLE 3
Tightly Bound Magnesium Concentration - Comparison
of Women Presenting and Women Not Presenting
Preeclampsia During Pregnancy

| | |
|---|---|
| Preeclamptic | Not Preeclamptic |

(Microgram Magnesium/0.50 mg. Membrane Protein)

| | |
|---|---|
| 0.515 | 0.678 |
| 0.532 | 0.630 |
| 0.530 | 0.598 |
| 0.519 | 0.619 |
| 0.475 | 0.667 |
| 0.541 | 0.681 |
| Average = 0.519 ± 0.010 | Average = 0.646 ± 0.014 |

P value (students “t” test) = P << 0.01

### Example 5

### Remediation of Magnesium Binding Defect with In Vitro Administration of Peptides to Erythrocytes from Individuals Who Presented Preeclampsia

The effect of the tetrapeptide (SEQ ID NO:2) and pentapeptide (SEQ ID NO:1) on the binding of magnesium in the erythrocyte membrane from individuals who presented preeclampsia can be demonstrated by measuring the magnesium contents of ghosts after the erythrocytes are incubated with one or both of the peptides of SEQ ID NO:1 and SEQ ID NO:2. For this purpose, approximately 5 ml samples of heparinized blood are used. After the plasma is separated from the erythrocytes, the erythrocytes are washed twice with 10 ml of cold erythrocyte wash. Approximately 3 mg of the desired peptide is then added to the erythrocytes from the preeclamptic subjects and the mixture is incubated for approximately 3 hrs. at 37° C., chilled on ice and the erythrocyte ghosts prepared and analyzed for magnesium and protein as described in Example 1. The magnesium concentration of the incubated erythrocyte samples can then be compared with erythrocytes which were not incubated, or other standard, to assess the remedial effect of the peptides.

### Example 6

### Generation of Plasma Membrane Tightly Bound Magnesium Deficient Erythrocytes

In order to identify those substances which promote binding of magnesium ion to plasma membranes (tightly bound magnesium ion) and that ameliorate or correct the magnesium binding defect, a deficit of membrane tightly bound magnesium was created experimentally in erythrocytes without the magnesium binding defect. Human erythrocytes were obtained from blood samples that had been drawn two days previously and kept for varying periods of time at room temperature, or at approximately 5° C. Preferred results were obtained with those samples in which either sodium citrate or sodium heparin was the anticoagulant. The length of time held and time held before moving to room temperature or 5° C is not believed to be critical. Although it was possible to create the MgBD in cells held as above, more reproducible results would be expected if fresh drawn blood were to be used.

The following procedure was employed to generate a deficit of plasma membrane tightly bound magnesium ion in the erythrocytes. Art recognized adaptations of this procedure can be used for generation of a deficit in other somatic cells, such as from urine or buccal cells. Samples of blood described above were kept at room temperature overnight and then pooled in a beaker placed in crushed ice and the blood was stirred slowly to collect any fibrin that may have formed. The blood was then centrifuged (600×g, 15 minutes) at 5° C. and the plasma layer removed by aspiration. Residual plasma was removed from the cells by suspending them twice in equal volumes of cold normal saline and then twice in equal volumes of cold Alsever's solution (trisodium citrate 32.5 mM, citric acid 2.7mM, D-glucose 114 mM, NaCl 137 mM, pH 6.1). Finally, the washed cells were suspended again in an equal volume of the same Alsever's solution to which approximately 1.25 mg/ml of sodium deoxycholate was added. This suspension was filtered through cheese cloth and kept at 4° C. with gently mixing once each day. Although the Alsever's plus sodium deoxycholate solution is a preferred example of a holding solution, any cell stabilizing buffer plus sodium deoxycholate may be utilized in the practice of the invention.

The concentration of magnesium ion tightly bound to the erythrocyte membranes was determined by using 5 mL portions of the approximately two day old blood sample and the stored erythrocyte suspension. After these portions were centrifuged as above and the supernatant fluids aspirated, the residues of cells were suspended four times in 15 mL portions of cold hemolyzing buffer (Tris Tris HCl 5 mM, sucrose 14 mM, pH 7.4) and centrifuged (15,000×g, 15 minutes). The supernatant fluids were removed by gentle aspiration. The decanting procedure was repeated as necessary to remove all of the dark red residue (Example 1). The penultimate residue was resuspended by gently pouring the hemolyzing buffer onto the residue and decanting the readily suspended cell membranes from the small, dark red, dense residue which adhered to the centrifuge tube. The final, nearly colorless residue of membranes was transferred and brought to a total volume of 25 mL in a volumetric flask using the hemolyzing buffer. These preparations of erythrocyte membranes were stored at 4° C. until the total protein contents were determined by the Bicinchoninic Acid procedure and the total magnesium contents determined by atomic absorption spectroscopy. From these values the concentrations of tightly bound magnesium in the cell membranes were expressed as nanograms of magnesium per 0.50 mg of membrane protein. This determination of the magnesium binding was repeated each day or so until the level of binding first ceased to decrease. Table 4 shows the levels of tightly bound magnesium in aged erythrocyte membranes.

TABLE 4
Tightly Bound Magnesium Levels in Aged Erythrocytes

| | Aging | | |
|---|---|---|---|
| Age of Cells⁺ | Medium | Magnesium Binding* | P** |
| 2 | Plasma | 621 ± 10.0 (4) | |
| 4 | Alsever's | 514 ± 40.0 (4) | <0.05 |
| | Solution | | |
| 8 | Alsever's | 391 ± 27.0 (4) | <0.05 |
| | Solution | | |
| 10 | Alsever's | 414 ± 14.0 (4) | NS |
| | Solution | | |

⁺Days since blood was drawn
*Nanograms of magnesium per 0.50 mg of membrane protein. Number of analyses in parentheses.
**Comparison of adjacent binding values

Under the experimental conditions described herein, the magnitude of the magnesium binding defect increased by about 100 ngm per 0.50 mg of membrane protein per day of aging. These results with erythrocytes suggest that the defect probably becomes constant after approximately six days of aging in the in vitro system. To be noted is the relatively large residual concentration of magnesium ion that remained bound to the erythrocyte membrane which amounted to about 50 percent of the concentration present in the erythrocytes from freshly drawn blood. The response of other somatic cells may vary in timing, or otherwise, however the objective of this method is to measure tightly bound magnesium ion over time until a significant reduction is observed, and the cells remain intact.

Preparations of aged erythrocytes in which the MgBD has been developed were used as soon as possible in incubations with normal blood plasma, and other magnesium containing fluids, in search of substances which could correct the MgBD (Example 7). For this purpose 5 mL portions of the suspensions of aged erythrocytes were centrifuged (600×g, 15 minutes) and the supernatant fluids aspirated. The residues of cells were then suspended twice in 10 mL of cold normal saline, as described above. Then 5 mL portions of the incubation substances being tested were added to the cell residues. These mixtures were incubated at 37° C. for three hours. Afterwards, the concentrations of the tightly bound magnesium in the cell membranes were determined as described above in Example 1.

### Example 7

### Promotion of Magnesium Binding in Magnesium Deficient Erythrocytes

Magnesium deficient erythrocytes, prepared as in Example 6, were used in the in vitro method of the present invention to detect substances which promote the binding of magnesium ions to the plasma membranes of somatic cells (tightly bound magnesium). Substances which can be screened for promotion of magnesium binding include, but are not limited to, peptides and peptide mimetics. Table 5 shows the results of quadruplet incubations in which the incubation medium was selected from: normal saline, freshly prepared normal human blood plasma, Krebs-Ringer phosphate glucose (KRPG) (NaCl 120 mM, KCl 4.8 mM, CaCl₂ 0.6 mM, MgSO₄ 1.2 mM, D-glucose 5.5 mM, NaH₂PO₄ 2.9 mM, Na₂HPO₄ 12.5 mM, pH 7.4), or KRPG medium which contained six micrograms of Substance P. Also shown are the results of duplicate incubations performed at the same time in which the incubation medium was 5 mL portions of KRPG which contained commercial preparations of amidated peptides derived from the N-terminal and C-terminal regions of Substance P (Sigma Chemical Co., St. Louis, Mo.). In each case, the amount of peptide added to the 5 mL of medium was that amount which was theoretically obtainable from six micrograms of Substance P.

Results with Krebs-Ringer phosphate glucose (KRPG) show activity comparable to those obtained with normal saline. However, this uptake appears to have been due to a slight degeneration of the structure of the erythrocyte membrane which apparently exposed anionic structures in the membrane protein. This loss of membrane structure continued as the erythrocytes aged until this cause for uptake accounted for all of the magnesium ion uptake. This total uptake was more than the total of the initial membrane content. Presumably, this structural change is minimal until those ions concerned with the magnesium binding defect are lost. Thus the magnesium ions concerned with the MgBD appear to have a structural function consistent with their ability to control the entrance of magnesium ions into the cell.

Consequently, only those uptakes of magnesium ions in excess of that occurring in the presence of KRPG alone are presented in Table 5 as magnesium ion binding. With this qualification, only normal plasma, Substance P (SEQ ID NO:3), 7-11 Substance P (SEQ ID NO:1) and 8-11 Substance P (SEQ ID NO:2) promoted measurable binding of magnesium ions to the erythrocyte membrane. These substances can be expected to promote binding to the plasma membranes of all other somatic cells. Among these materials, the approximate ratios of binding activities are, in the order the materials are listed, 1:1.1:3.7:4.6. It is of interest that equal molar amounts of Substance P, the 7-11 Substance P pentapeptide (Phe-Phe-Gly-Leu-Met-NH₂), and the 8-11 Substance P tetrapeptide (Phe-Gly-Leu-Met-NH₂) were tested. Consequently, the results show that the pentapeptide and tetrapeptide were about four times as active as Substance P. Of further note is the fact that the peptides 1-9 Substance P, and 9-11 Substance P showed no measurable activity in this test.

TABLE 5
Promotion of Magnesium Binding in Magnesium
Depleted Erythrocyte Membranes

| Incubation Medium | Magnesium Binding⁺ | P* |
|---|---|---|
| 0.9% saline | 414 ± 14 (4) | |
| Krebs-Ringer phosphate | 520 ± 9 (4) | <0.01 |
| Glucose (KRPG) | | |
| Normal plasma | 613 ± 24 (4) | <0.01 |
| KRPG + 6 ugm Substance P | 624 ± 16 (4) | NS |
| KRPG + 1-9 Substance P | 500, 490 | |
| KRPG + 7-11 Substance P | 886, 866⁺⁺ | |
| KRPG + 8-11 Substance P | 968, 954⁺⁺ | |
| KRPG + 9-11 Substance P | 546, 510 | |

⁺Nanograms of magnesium per 0.50 mg of membrane protein
*Comparison of adjacent values
⁺⁺Comparison to KRPG values alone

In summary, pentapeptide (SEQ ID NO:1) and tetrapeptide (SEQ ID NO:2) are significantly more active than Substance P in promoting binding of magnesium. While not wanting to be bound by any particular theory of action, the discoveries reported herein indicate that the molecular configuration of the pentapeptide (SEQ ID NO:1) and tetrapeptide (SEQ ID NO:2) are significantly different from that of Substance P which may explain the reported differences in their effect on the magnesium binding defect and blood pressure in the experimental animals.

### Example 8

### Relationship of the MGBD to the Occurrence of Salt-sensitive Essential Hypertension in Individuals with Type 2 Diabetes Mellitus

Increased insulin resistance has previously been observed to occur in some salt-sensitive hypertensive patients (Resnick et al., 1990). However, a relationship between the occurrence of the MgBD and the occurrence of type 2 diabetes mellitus itself has not previously been reported.

Twenty-four type 2 diabetics were randomly selected and screened by the use of a brief questionnaire and a blood pressure measurement. All were normotensive, nonmedicated, generally more than 40 years of age, and mildly diabetic as indicated by hemoglobin A_{1c} (HbA_{1c}), i.e., HbA_{1c} levels and fasting blood glucose levels of not greater than 180 mg/dL. Control volunteers were recruited from the hospital personnel and screened as above. Twenty-five subjects were chosen that had similar characteristics as the selected diabetic subjects except they had no signs or symptoms of diabetes.

There were no significant differences between the magnesium binding values of the males and females within the control and diabetic groups. While the average ages of the individuals in the control and diabetic groups were different, the ranges of ages in the two groups were very similar. The age range of the group members correlate with the clinical identification of overt type 2 diabetes mellitus in patients. An Hb A_{1c} level of less than 6.0 percent is generally interpreted as an indicator of the absence of diabetes. Twenty-three of the controls satisfied this criterion while one subject's value was not recorded and another's value was marginally greater than 7.0 (range, nondiabetic adults, 4.5-6.5 percent). Six of the twenty-four diabetic subjects tested had Hb A_{1c} levels in the “near normal” range of six-to-seven percent. Since the magnesium binding values for these control and diabetic subjects were very similar to those of other members of their groups, the initial classifications of these individuals were maintained. Of the type 2 diabetes mellitus patients involved in the present study, all were normotensive and had the MgBD. In a previous study (Mattingly et al., 1991) of 21 other individuals, all of which were observed to be type 2 diabetics, some were also observed to be hypertensive. Thus, it was concluded that the MgBD is the cause of type 2 diabetes but is only a risk factor for salt-sensitive essential hypertension.

The procedure used to determine the tightly bound magnesium ion concentrations in the erythrocytes from these subjects is as described above (Example 1). Table 6 shows the characteristics of the subjects studied and the values for the tightly-bound magnesium ion concentrations in their erythrocyte membranes.

TABLE 6
Type 2 Diabetes Mellitus Subjects Studied

| Quantity | | | | | Observed | |
|---|---|---|---|---|---|---|
| Measured | Subjects | Male/Female | Number | Mean ± SEM | Range | P |
| Age* | Controls | 6/19 | 25 | 48.9 ± 2.01 | 21-79 | <0.02 |
| | Diabetics | 18/4 | 22 | 56.6 ± 2.30 | 38-70 | |
| HbA_{1c}** | Controls | 5/19 | 24 | 5.5 ± 0.12 | 4.9-7.9 | <0.01 |
| | Diabetics | 19/4 | 23 | 8.7 ± 0.56 | 5.2-15 | |
| MG Binding⁺ | Controls | 6/19 | 25 | 700 ± 12 | 594-788 | <0.01 |
| | Diabetics | 20/4 | 24 | 465 ± 12 | 343-567 | |

*Years
**Percent
⁺Nanograms of magnesium per 0.50 mg of membrane protein

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

List of References

Allen and Greer (1995) “Calcium and Magnesium Metabolism Beyond Infancy” in Calcium and Magnesium Metabolism in Early Life, Tsang (ed), CRC Press, pp. 136-138

Arbogast et al. (1994) Lancet 343:341-341

Bradford M M (1976) Anal. Biochem 72:248 -254

Caruso et al. (1999) Hum Reprod 14:219-223

Chard T (1990) “An Introduction to Radioimmunoassay and Related Techniques”, (4^{th} ed.), Elsevier Science Publishing Co., Inc, New York

Cooper et al. (1993) Hyperntens Pregnancy 12:1-23

Corica et al. (1996) Biol Trace Elem Res 51:13-21

Davey and MacGillivray (1988) Am J Obstet Gynecol 158:892-898

Dawson et al. (1962) Data for Biochemical Research, Oxford University Press, pp. 208-209

Dekker and Sabai (1998) Am J Obstet Gynecol 179:1359-1375

de Mondonca et al. (1985) Klin. Wochenschr. 63 (Suppl 3):66-69

de Valk et al. (1998) Diabet Med 15:503-507

Eibl et al. (1995) Diabetes Care 18:188-192

Eriksson and Kohvakka (1995) Ann Nutr Metab 39:217-223

Gifford et al. (1990) Am J Obstet Gynecol 163:1689-1712

Goding (1986) Monoclonal Antibodies: Principles and Practice (2d ed.), Academic Press, NY pp. 60-61

Harlow and Lane (1988) Antibodies: A Laboratory Manual (Cold Spring Harbor Laboratory, Cold Spring Harbor, N.Y.)

Holvoet et al. (1994) J Clin Invest 93:89-98

Johnson et al. (1993) J Virol 66:2952-2965

Kaaja et al. (1990) Acta Obstet Gynecol Scand 69:393-396

Kaaja et al. (1995) Obstet Gynecol 85:353-356

Kuby (1991) Immunology (2 d ed.), W. H. Freeman and Co., pp 135-139

Lehninger A L (1975) Biochemistry (2^{nd} ed.), Worth Publishers, p. 390

Lorentzen B et al. (1998) Acta Obstet Gynecol Scand 77:22-27

Martinez Abundis E et al. (1996) Am J Hypertens 9:610-614

Mattingly et al. (1991) Clin Exper Hyper-Theory and Practice A13:65-82

Meyer and Marche (1988) Am. J Med. Sci. 295:396-399

Ness and Roberts (1996) Am J Obstet Gynecol 175:1365-1370

Qu and Stuesse (1990) Peptides 11: 955-961

Rayson B M (1988) J Biol Chem 263:11056-11058

Redman C (1991) Semin Perinatol 15:257-262

Redman et al. (1999) Am J Obstet Gynecol 180:499-506

Resnick et al. (1990) Hypertension 17:951-957

Rendina G (1971) Experimental Methods in Modern Biochemistry. W. B. Saunders Co. p 161

Roberts J (1998) Semin Reprod Endocrinol 16:5-15

Roberts et al. (1998) Br J Obstet Gynaecol 105:1095-1100

Rosati et al. (1988) Hypertension 11:41-48

Sibai, B M (1996) New Eng. J Med. 335:257-265.

Searcy R L (1961) Diagnostic Biochemistry, McGraw Hill Book Co., pp 373-380

Smarason et al. (1993) Br J Obstet Gynaecol 100:943-949

Wallach et al. (1962) J. Lab. Clin. Med. 59:195-210

Wells and Agrawal (In Press) Biol Trace Elem Res

Wells and Agrawal (1992) Can. J. Physiol. Pharmacol. 70:1225-1229

Wells and Blotcky (2001) Can. J. Physiol. Pharmacol 79:779-784

Patents and Patent Applications:

U.S. Pat. No. 4,196,265

U.S. Pat. No. 4,376,110

U.S. Pat. No. 4,486,530

U.S. Pat. No. 6,309,888

U.S. Pat. No. 6,372,440

U.S. Pat. No. 6,392,020

U.S. Pat. No. 6,455,734

What is claimed is:

1. A method of assessing a predisposition to a physiological disorder associated with magnesium binding defect in an individual, compising: measuring the level of peptide in a sample of body fluid of said individual, said peptide comprising one or more of amino acid sequence set forth in SEQ ID NO:1, or amino acid sequence set forth in SEQ ID NO:2, or amino acid sequence set forth in SEQ ID NO:4, and comparing said level of peptide to a standard, whereby a significantly lower level of said peptide is indicative of a predisposition of said individual to said physiological disorder.

2. The method of claim 1 wherein said predisposition to a physiological disorder associated with magnesium binding defect is a predisposition to presenting preeclampsia during pregnancy.

3. The method of claim 2 wherein the level of said peptide in said sample is measured by an immunological assay that can indicate the presence of one or more of amino acid sequence set forth in SEQ ID NO:1, or amino acid sequence set forth in SEQ ID NO:2, or amino acid sequence set forth in SEQ ID NO:4.

4. The method of claim 3 wherein said immunological assay utilizes a monoclonal antibody.

5. The method of claim 4 wherein said monoclonal antibody cross reacts with each of said peptides.

6. The method of claim 3 wherein said immunological assay is an enzyme-linked immunosorbent assay, and said sample of body fluid is blood.

7. The method of claim 1 wherein said predisposition to a physiological disorder associated with magnesium binding defect is a predisposition to salt-sensitive essential hypertension.

8. The method of claim 7 wherein the level of said peptide is measured by an immunological assay that can indicate the presence of one or more of amino acid sequence set forth in SEQ ID NO:1, or amino acid sequence set forth in SEQ ID NO:2, or amino acid sequence set forth in SEQ ID NO:4.

9. The method of claim 8 wherein said immunological assay utilizes a monoclonal antibody.

10. The method of claim 9 wherein said monoclonal antibody cross reacts with each of said peptides.

11. The method of claim 8 wherein said immunological assay is an enzyme-linked immunosorbent assay.

12. The method of claim 7, further wherein said method distinguishes between salt-sensitive essential hypertension and salt-resistant essential hypertension disorders in an individual.

13. The method of claim 1 wherein said predisposition to a physiological disorder associated with magnesium binding defect is a predisposition to type 2 diabetes mellitus associated with the magnesium binding defect.

14. The method of claim 13 wherein the level of said peptide is measured by an immunological assay that can detect the presence of one or more of amino acid sequence set forth in SEQ ID NO:1, or amino acid sequence set forth in SEQ ID NO:2, or amino acid sequence set forth in SEQ ID NO:4.

15. The method of claim 14 wherein said immunological assay utilizes a monoclonal antibody.

16. The method of claim 15 wherein said monoclonal antibody cross reacts with each of said peptides.

17. The method of claim 14 wherein said immunological assay is an enzyme-linked immunosorbent assay.

18. The method of claim 13, further wherein said method distinguishes between lipid-induced type 2 diabetes mellitus and type 2 diabetes mellitus associated with magnesium binding defect in an individual.

19. A method for monitoring progress in treatment of a physiological disorder associated with magnesium binding defect in an individual, comprising:
a. measuring the level of peptide in a sample of body fluid of said individual, said peptide comprising one or more of amino acid sequence set forth in SEQ ID NO:1, or amino acid sequence set forth in SEQ ID NO:2, or amino acid sequence set forth in SEQ ID NO:4;
b. comparing said level of peptide to the level of said peptide after treatment, whereby a significant increase in the level of said peptide is indicative of the progress of treatment of said individual.

20. A monoclonal antibody that specifically binds to a peptide or its peptide mimetic comprising:
a. one or more of amino acid sequence set forth in SEQ ID NO:1;
b. amino acid sequence set forth in SEQ ID NO:2;
c. amino acid sequence set forth in SEQ ID NO:4.

21. A prognosis reagent for determining presence of magnesium binding defect, comprising the monoclonal antibody of claim 20.

22. A method for generating a deficit of plasma membrane tightly bound magnesium ion in mammalian somatic cells, comprising:
a. obtaining a sample of body fluid comprising somatic cells;
b. collecting said somatic cells from said body fluid by centrifugation;
c. resuspending the somatic cells in a cell stabilizing buffer including about 1 to 1.5 mg/ml sodium deoxycholate;
d. removing a sample of said suspended somatic cells;
e. measuring the level of tightly bound magnesium ion in said sample of the somatic cells; and
f. repeating steps d. and e. at subsequent times until said level of tightly bound magnesium is significantly reduced and the somatic cells remain intact.

23. The method of claim 22 wherein said body fluid is human blood and said somatic cells are erythrocytes, and further, wherein an anticoagulant is added to said sample of human blood.

24. The method of claim 23, wherein said anticoagulant is sodium heparin or sodium citrate, and said cell stabilizing buffer without magnesium is Alsever's solution.

25. A method for identifying substances which promote binding of tightly bound magnesium ion to a plasma membrane of mammalian somatic cells, comprising:
a. suspending mammalian somatic cells having a deficit of plasma membrane tightly bound magnesium in a physiological medium, including magnesium ion;
b. adding a substance to be tested to said suspension;
c. measuring the level of tighly bound magnesium ion in the plasma membrane of the somatic cells of step b.,
wherein a significant increase in the level of plasma membrane tightly bound magnesium after addition of the substance to be tested is indicative of promotion of binding by said substance.

26. The method of claim 25, wherein said mammalian somatic cells having a deficit of plasma membrane tightly bound magnesium are generated by the method of claim 26.

27. A monoclonal antibody that specifically binds to a substance identified by the method of claim 25 as a substance which promotes binding of tightly bound magnesium ion.

28. The method of claim 26, wherein said somatic cells are erythrocytes and said physiological medium is Krebs mammalian Ringer solution, including glucose.

29. A method for ameliorating or correcting a magnesium binding defect in an individual, comprising administering to said individual a substance identified by the method of claim 25 as a substance which promotes binding of tighly bound magnesium ion to the plasma membrane of mammalian somatic cells.

30. The method of claim 29, wherein said promoter is a peptide or its peptide mimetic, wherein said peptide comprises amino acid sequence set forth in SEQ ID NO:1, or amino acid sequence set forth in SEQ ID NO:2, or amino acid sequence set forth in SEQ ID NO:4.
